(19) United States
(12) Patent Application Publication
(10) Pub. No.: US 2006/0160085 A1
(43) Pub. Date: Jul. 20, 2006

(54) Novel buffer formulations for isolating purifying and recovering long-chain and short-chain nucleic acids

(76) Inventors: Timo Hillebrand, Honow (DE); Peter Bendzko, Berlin (DE)

Correspondence Address: Buchanan Ingersoll, One Oxford Centre, 301 Grant St.., 20th Floor, Pittsburgh, PA, 15219, US

(21) Appl. No.: 10/534,387

(22) PCT Filed: Nov. 10, 2003
(86) PCT No.: PCT/DE03/03728

§ 371 Date: Nov. 22, 2005

(30) Foreign Application Priority Data
Nov. 8, 2002 (DE) 102 52 545.5
Nov. 14, 2002 (DE) 102 53 351.2

(51) Int. Cl. C12Q 1/68; C07H 21/04

(52) U.S. Cl. 435/6; 536/25.4

(57) ABSTRACT

The invention relates to novel formulations of buffers used for isolating, purifying and recovering long-chain and short-chain nucleic acids. The areas of application of the inventive method include all laboratories engaged in isolating nucleic acids, such as laboratories used in forensic medicine, food diagnosis, medical diagnosis, molecular biology, biochemistry, genetic engineering and all other related fields. The inventive method is characterized in that the solution containing the nucleic acid is prepared with additives whereby containing monovalent and multivalent cations as well as an alcohol and, optionally, additional additives. The solution is subsequently brought into contact with the solid phase, whereupon the support is optionally washed, and the nucleic acid is removed from the solid phase or the solution optionally contains multivalent and/or monovalent cations, optionally one alcohol, and optionally contains additional additives, and a specific pH value is set between 7 and 10. Ammonium chloride, sodium chloride and/or potassium chloride are used as monovalent salt components. Magnesium chloride, calcium chloride, zinc chloride and/or manganese chloride are used as multivalent salt components. A particularly preferred variant involves the use of identical molar amounts of sodium chloride and manganese chloride.

The invention relates to a novel formulation of buffers for isolating, purifying, and recovering long- and short-chain nucleic acids.

The method's areas of application include all laboratories engaged in isolating nucleic acids, such as those dealing with forensic medicine, food diagnosis, medical diagnosis, molecular biology, biochemistry, genetic engineering, and all other related fields.

Under classic conditions, DNA is isolated from cells and tissue whereby the base materials are dissolved under highly denaturing and reducing conditions, partially also through the use of protein-digesting enzymes. The emerging nucleic acid fraction is cleaned by means of phenol-/chloroform extraction steps, and the nucleic acids are obtained through dialysis or ethanol precipitation from the aqueous phase (Sambrook, J., Fritsch, E. F. and Maniatis, T., 1989, CSH, “Molecular Cloning”).

This “classic procedures” to isolate nucleic acids from cells and especially from tissue are very time-consuming (sometimes longer than 48 hours), require a considerable amount of sophisticated equipment, and can also not be carried out in field conditions. Besides, such methods pose a not insignificant health hazard due to the use of chemicals such as phenol and chloroform.

Various alternative procedures to isolate nucleic acids from different biological base materials allow one to circumvent the time-consuming and toxic phenol-/chloroform extraction from nucleic acids and reduce the time spent in carrying out the process.

All these procedures are based on a method developed and first described by Vogelstein and Gillespie (Proc. Nat'l Acad. Sci., US, 1979, 76, 615-619) regarding the preparative and analytic purification of DNA fragments from agarose gels. The method combines the dissolution of the agarose containing the DNA strand to be isolated in a saturated

solution of chaotropic salt (NaI) with a binding of the DNA on glass particles. The DNA affixed to the glass particles is then washed with a washing solution (20 mM tris-HCl [pH 7.2]; 200 mM NaCl; 2 mM EDTA; 50% v/v ethanol) and finally removed from the carrier particles.

Until now, this method experienced a series of modifications and is currently used for various procedures pertaining to the extraction and purification of nucleic acids from diverse sources (Marko, M. A., Chipperfield, R. and Birnboim, H. G., 1982, Anal. Biochem., 121, 382-387).

In addition, there are currently numerous reagent systems worldwide, especially for the purification of DNA fragments from aragose gels and the isolation of DNA plasmid from bacterial lysates, as well as for the isolation of long-chain nucleic acids (genomic DNA, cellular total RNS) from blood, tissue, or also cell cultures.

All these commercially available kits are based on the conventionally known principle of nucleic acids binding on mineral carriers given the presence of solutions of various chaotropic salts and using suspensions of finely ground glass powder (e.g. Glasmilk, BIO 101, La Jolla, Calif.), diatomaceous earth (Sigma) or also silica gels (Diagen, DE 41 39 664 A1).

A procedure to isolate nucleic acids practical for many different applications is portrayed in U.S. Pat. No. 5,234,809 (to Boom). It describes a process to isolate nucleic acids from nucleic acid-containing base materials through the incubation of the base material with a chaotropic buffer and a DNA-binding solid phase. The chaotropic buffers carry out both the lysis of the base material as well as the binding of the nucleic acids to the fixed phase. The process is well suited to isolate nucleic acids from small sample quantities and in particular it has a practical application in the field of isolating viral nucleic acids.

Significant disadvantages of the process are, among other things, that the dissolving carried out by the chaotropic buffers is not usable for all materials, or is only extremely inefficient for larger quantities of base materials and can only be completed in a very time-consuming way. In addition, mechanical homogenization procedures

are required, if for example DNA is to be isolated from tissue samples. Furthermore, different problems require the use of various chaotropic buffers of continuously different concentrations. The process is thus in no way universally usable.

The physical-chemical principle of today's commercially available systems, according to known prior art, used to isolate nucleic acids based on the binding of nucleic acids to the surfaces of mineral carriers consists of disturbing the superordinate structures of the aqueous environment through which the nucleic acids adsorb on the surface of mineral materials, especially glass or silica particles. The disturbance of the superordinate structures of the aqueous environment always results under the presence of chaotropic ions and is almost quantitative at high concentrations of these. Given this described physical-chemical basis, all commercially available systems for the isolation of nucleic acids contain buffer compounds with highly ionic chaotropic salts for the binding of nucleic acids to a nucleic acid binding solid phase.

Specific modifications of these processes pertain to the use of specific carrier materials that exhibit application-related advantages for certain tasks (see, for example, Invitek GmbH WO 95/34569), yet have the same disadvantages.

A commonality of all the described processes for the isolation of nucleic acids by means of binding the nucleic acids to mineral solid phases using chaotropic saline solutions is that for the binding of nucleic acids to the selected carrier materials, high concentrations of carrier materials must be utilized. Such chaotropic salts (e.g. guanidinisothiocyanate, guanidine hydrochloride, sodium perchlorate or sodium iodide) are highly toxic substances. The highly ionic buffer systems utilized often result in a transfer of salt contamination into the nucleic acids to be isolated and are often the reason that a series of downstream applications (PCR, restricted digestion, hybridization, and ligations) cannot be carried out and if so only partially. In addition, when dealing with chaotropic buffers there is a considerable health hazard (especially with long-term exposure) as well as substantial environmental damage due to sewage-borne pollutants.

Patent DE 198 56 064 describes a first-ever innovative process to isolate nucleic acids. According to it, the binding of the nucleic acids to be isolated to mineral carriers results without the use of previously required highly ionic chaotropic salts. The process is based (as are the chaotropic processes) on the lysis of the base material, the binding of the nucleic acid to a mineral carrier material, the subsequent washing of the bound nucleic acids with ethanol-containing washing buffers, the removal of ethanol and the final elution of the nucleic acids with a weakly ionic elution buffer or water.

For special protocols, e.g. the isolation of PCR fragments from amplification formulas, the lysis step is not required. The PCR reagent formula is mixed with a required binding buffer and incubated with a mineral carrier material. There then follows washing with an ethanol-containing washing buffer, then the removal of ethanol, and finally the elution of the bound nucleic acid from the carrier material.

That the processes without the use of chaotropic buffers have obvious advantages is demonstrated by the fact that after this initial description, other patents (e.g. DE 100 33 991) describe the potential advantages of this new binding chemistry.

Interestingly, it appears that all commercially available systems worldwide for the isolation of nucleic acids based upon the binding of nucleic acids to mineral carrier materials (magnetic particles, membranes, carrier suspensions, among others) operate primarily according to the described process. Since the initial description by Vogelstein and Gillespie, the bound nucleic acids are always washed with alcohol or acetone-containing saline solutions. The washing steps are an essential component of the extraction protocol and besides the removal of bound undesirable inhibitory materials, they also always serve in the necessary removal of the salts required for the binding of the nucleic acids.

However, the use of alcohol or acetone-containing washing buffers always signifies a quite substantial disadvantage unsolved to date. It is known that even traces of alcohol in the final nucleic acid can substantially impair downstream applications. For that reason, it is always necessary to integrate an ethanol-removal step in the actual process to isolate or purify nucleic acids.

This is always problematic when magnetic particles or particular carrier suspensions are used, the process requires considerable time, and can

result in the irreversible loss of nucleic acids, especially in the over-drying of carrier materials.

The step of removing residual alcohol is especially problematic for applications that isolate nucleic acids in high throughput ranges.

Generally, up to 30 minutes are required within the scope of automated nucleic acid purifying processes to free membranes in filter disks or magnetic particles of the residual alcohol.

In addition, the actual washing steps with alcoholic wash buffers are very time consuming and obviously also cost-intensive especially in high throughput ranges.

From the disadvantages of the prior art, the task emerges to avoid the use of alcoholic components and to thereby achieve a substantial shortening of the isolation and purification process.

The invention is realized according to the claims.

Based on the use of non-chaotropic buffer formulas, as already described in patent DE 198 56 064, one can see that only unexpectedly low concentrations are required for the binding of nucleic acids to be isolated.

The invention's core point is in the simultaneous use of mono- and multivalent cations, preferably divalent cations, for the binding of nucleic acids to the solid phase.

Na⁺, K⁺ and NH⁺ in the form of the corresponding salts are preferred as monovalent cations. Mg⁺², Ca⁺², Zn⁺² and Mn⁺² in the form of corresponding salts are preferred as divalent cations. An especially preferred embodiment is the combined use of Na⁺ and Mg⁺².

According to the invention, the mono- and multivalent cations, preferably divalent cations, can be used in diverse quantity ratios. Successful results are best achieved in the broad range of mono-/divalent cations in a molar ratio of 9:1 to 1:9. Ratios of 7:3 to 3:7 and 6:4 to 4:6 are preferred. Especially preferred are the embodiments with identical (1:1) or almost identical molar masses of mono- and divalent cations.

The total cation concentration in the solution prior to binding to the solid phase is preferably <0.5M.

If the nucleic acids are present in a solution that already contains mono- or divalent cations, e.g. after a preceding lysis of diverse base materials, then the present amount of cations is taken into account in adjusting the optimal cation concentration according to the invention. Thus, if the lysis buffer contains divalent cations that will be contained in the solution after lysis, then only the required amount of monovalent cations is added (or vice versa).

An especially important characteristic of the invention is also that the washing buffers used according to the invention contain no alcoholic components, as was the case in all other prior art processes.

Surprisingly, in the combination of salts of a monovalent cation with a multivalent one that lie in a concentration range required for binding, the buffer formulations consisting of only one salt each are no longer sufficient for the binding to occur. Thus, the combination of magnesium chloride and sodium chloride at quantities of less than 5 mM each allow the binding of nucleic acids of an additional magnitude spectrum (Example 1). The use of mixed salts having monovalent and multivalent cations as integral components of binding buffers for the binding of nucleic acids to mineral carriers has not been described to date. The surprising result now allows an entirely new strategy to isolate and purify nucleic acids from complex base materials. Since surprisingly only extremely low salt concentrations are required in binding buffers, it becomes possible to isolate nucleic acids from complex samples or solutions that contain a plurality of materials to be removed with innovative washing buffers without the previously required ethanol or also entirely without a washing step.

This has tremendous advantages for the isolation of nucleic acids and solves the described problems of using alcohol-containing washing buffers, especially for automated high throughput applications, in an ideal manner. Thus, the use of buffer formulations according to the invention allows, for example, the purification of PCR products made of complex PCR reagent formulas for a subsequently sensitive sequencing reaction and without a single washing step in the form of an automated application (binding of the PCR products to the filter membrane of a 96-gauge corrugated plate) in less than 10 minutes. Previous

procedures based on the binding of nucleic acids to a solid phase require about 45 minutes to 1 hour. In addition, the process sequence is also extremely simple and only consists of mixing the PCR starting solution with a binding buffer according to the invention, the transfer of the solution to the filter plate, the suctioning through of the solution, and the subsequent elution of the PCR products using water or a 10 mM tris-buffered aqueous solution. Thus, PCT products can be purified in an extremely timesaving, harmless, and cost-effective manner. The throughput can therefore be increased dramatically (also for less sophisticated equipment; e.g. a robot no longer requires a washing tool). The quality of the purified PCR products is very high, which one can see in the clean sequence reactions (Example 2).

Furthermore, surprisingly, one can see that nucleic acids having excellent quality and quantity can also be isolated from complex biological samples without washing steps or using an alcohol-less washing buffer. The isolation of a DNA plasmid described in an example in unexamined patent DE 100 33 991 without a washing step did not result from the previously manufactured, clarified lysate. The DNA plasmid to be isolated was first produced in a pure form by means of known standard procedures and this DNA plasmid was again incubated with a buffer, bound to a solid phase, and then after the necessary removal of the binding buffer's alcohol re-released from the solid phase.

With this invention, it is now possible to purify DNA plasmid directly from the clarified lysate, whereby again no washing with an alcohol-containing washing buffer is necessary, or the DNA plasmid can be isolated without a washing step directly after the resulting binding to a solid phase. The DNA plasmid is thus again of excellent quality and quantity (Example 3).

The invention also allows one to isolate genomic nucleic acids from complex biological samples in an extremely fast, cost-effective and simple manner. Accordingly, only a base material using for example a traditional protease K-digestion is dissolved by standard means in a buffer compatible for this purpose, then the lysated sample is mixed with a non-chaotropic binding buffer according to this invention, and the solution is incubated with a nucleic acid-binding solid phase, and if necessary washed with a non-alcoholic washing buffer (or if required, without a washing step) and then the genomic nucleic acid

is isolated from the solid phase using water or a tris-solution. The quality of the isolated DNA is again very high and can be used immediately for further applications.

Another variant of this invention results from the observation that the pH value of the binding buffer used has considerable influence on the yield of the nucleic acid to be produced and it possesses a selectivity in regard to the fragment lengths of the PCR products to be purified for example. It is not necessary to combine mono- and multivalent salts with each other in a solution. Divalent salts are preferred, and Mg or Ca salts are especially preferred for use.

This allows one to exclude selecting the pH value of binding buffers, e.g. small PCR products, from the isolation process. In addition, one can also see that surprising effects are achieved through the combination of salts and alcohol in the binding buffer in relation with the selection of the pH value. The alcoholic components in the binding buffer enable selectivity in regard to the size-related percentage of DNA fragments. The tendency is for a reduction of the binding buffer's pH value to reduce the yield of smaller DNA fragments up to complete inhibition of recovery. This is especially significant when heterogeneous sample mixtures are present.

Thus, for pH values >8, an almost quantitative recovery of DNA fragments from a broad size range (100 bp to 10,000 bp) can be achieved with binding buffers, if the binding buffer contains an alcoholic component.

For alcohol-containing binding buffers the pH value is set to 5-9.5 and preferably to 8-9.5, 6.5-8 or 5-6.5. Thus the recovery of certain fragment sizes is possible, i.e. certain fragment sizes are not recovered. It should be pointed out that the wording “no recovery” is not meant in the absolute sense, i.e. traces of nucleic acid fragments could always be recovered in a non-specific manner.

For alcohol-less binding buffers, recovery of DNA fragments results in quantitative amounts and over of a size range of 100 bp to 10,000 bp preferably at a pH value of >8.5. Finally, this invention allows one to develop binding buffers by means of combining salt components and alcohol components as well as modifying the pH value, which allow a selective recovery of certain nucleic acid fragments from heterogeneous base samples.

This invention thus universally permits a clear simplification of the process to isolate nucleic acids from complex nucleic acid-containing samples.

The process no longer requires toxic chemicals; the salt quantities used are dramatically reduced and clearly results in a less toxic impact on the environment; and the processes require less steps and are therefore significantly faster than previously used methods. Especially for high throughputs, there are now cost-effective and extremely fast processes available. The composition of the buffer formula also enables one to achieve a selective size-related percentage of DNA fragments to be recovered.

The invention is explained below using embodiments. The embodiments are not intended to represent any limitation to the invention.

### EMBODIMENTS

### Example 1

Purification of a range of DNA fragments from an aqueous solution; comparison of various binding buffers in regard to binding efficiency.

TH1 buffer (5 mM NaCl; 5 mM MgCl₂/tris HCl, isopropanol)

TH2 buffer (10 mM MgCl₂/tris HCl, isopropanol)

TH3 buffer (10 mM NaCl/tris HCl, isopropanol)

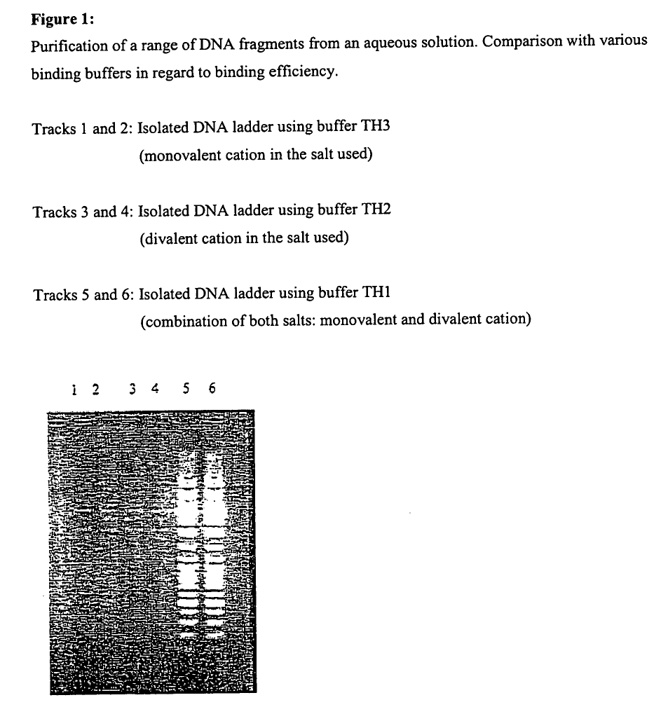

The TH1 buffer is a combination of a monovalent and divalent salt at a total strength of 10 mM. Buffers TH2 and TH3 each contain only one salt (with a monovalent cation and a divalent cation, respectively) for a total strength of 10 mM. 130 μl of the respective buffers were mixed with an aqueous 50-μl solution containing a commercial DNA ladder (Fermentas) and the solution is transferred to a centrifuge stand with a glass fiber fleece. Then, the solution was centrifuged for 1 minute at 10,000 rpm and the centrifuge column was transferred to a new reaction vessel. The elution of the bound fragments results by adding 30 μl of a 10 mM tris-HCl solution and subsequent centrifuging for 1 minute. The total time to isolate the DNA fragments was thus only about 2 minutes. The eluates obtained were applied to a 1.2% agarose gel and electrophoretically cut. As can clearly be seen in the electrophoretic image, efficient recovery of DNA fragments results using only the combination buffer. The single-salt containing binding buffers however only show a very slight binding ability (FIG. 1).

### Example 2

Purification of PCR products from a complex PCR reagent solution and subsequent use of the purified PCR products for DNA sequencing.

50 μl PCR reagent solutions were mixed with 130 μl of TH1 and TH4 binding buffers (50 mM NaCl; 50 mM MgCl2/tris HCl, isopropanol), then transferred to a centrifuge column with glass fiber fleece, centrifuged for 1 minute and finally the DNA is eluated from the column using 10 mM of tris HCl. The isolated PCR products were then

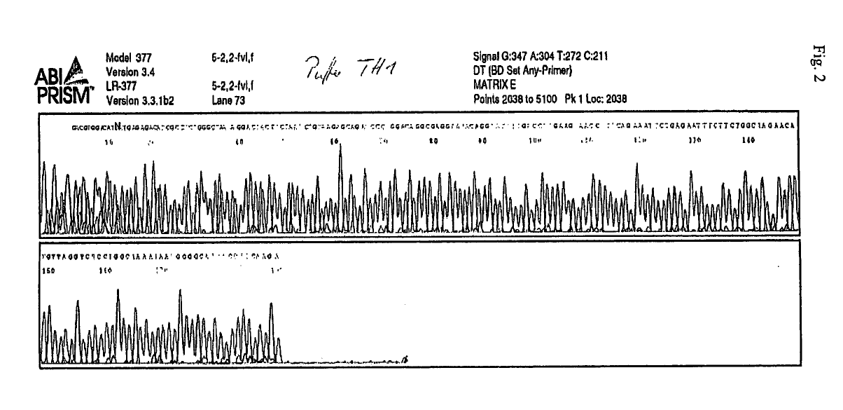

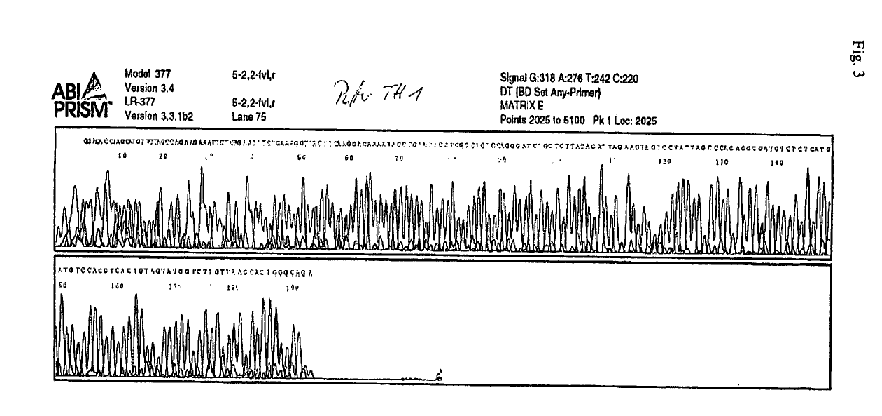

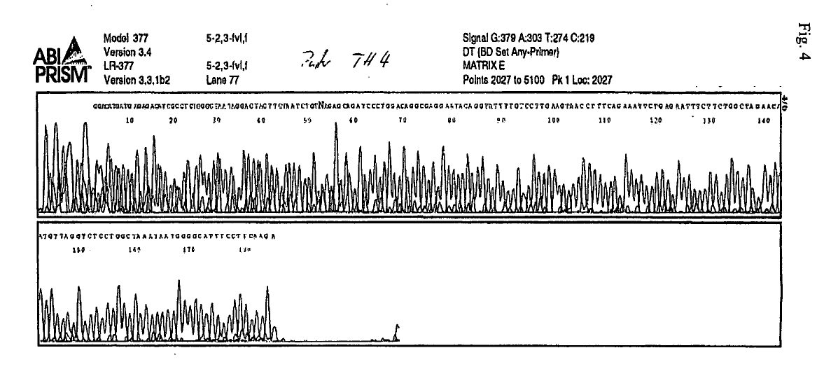

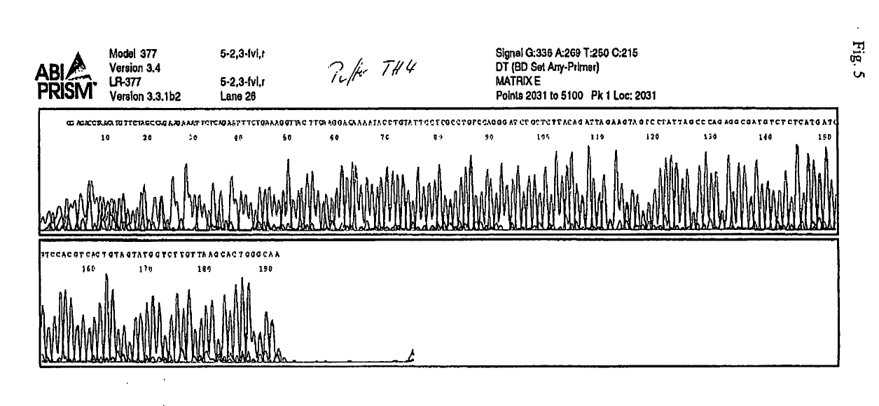

used for the sequencing. The sequencing results confirm that without using previously required wash steps, all interfering components were efficiently removed and high-purity DNA results (FIGS. 2-5).

### Example 3

Isolation of DNA plasmids from bacterial lysates. Comparison of the purity of the isolated DNA plasmids for various washing conditions or without a washing step.

2 ml of a bacterial overnight culture (XL-1 with pGEM plasmid) were centrifuged and the pellet was re-suspended with 200 μl of solution I (tris, EDTA Rnase A). Then, 200 μl of solution II (SDS/NaOH) were added. The reaction vessels were shaken carefully several times. Then 200 μl of a solution III (250 mM MgCl2/tris HCl) were added. The reaction vessels were carefully and briefly shaken and centrifuged for 5 minutes at maximum rpm. The clarified excess was mixed with 100 μl isopropanol and given to a centrifuge column with a glass fiber fleece and centrifuged for 1 minute. Then, each of the 3 samples was immediately mixed with 10 mM tris (no washing). 3 samples were mixed with 800 μl of an alcohol-less washing buffer (10 mM NaCl/10 mM MgCl2/tris HCl) and centrifuged for 1 minute and then the DNA plasmid was eluted with 10 mM tris-HCl from the column and 3 samples were washed with a 70% solution of ethanol. Then the ethanol was removed and the DNA plasmid was eluted again by adding 10 mM tris-HCl.

The following table illustrates the required preparation time, the quality and quantity of the isolated DNA plasmid.

| Sample | Washing step | Ration 260:280 | Yield | Preparation time |
|---|---|---|---|---|
| 1 | No washing | 1.71 | 12 μg | 8 min |
| 2 | No washing | 1.74 | 15 μg | 8 min |
| 3 | No washing | 1.81 | 14 μg | 8 min |
| 4 | Washing w/o | 1.82 | 15 μg | 10 min |
| | alcohol | | | |
| 5 | Washing w/o | 1.81 | 14 μg | 10 min |
| | alcohol | | | |
| 6 | Washing w/o | 1.84 | 12 μg | 10 min |
| | alcohol | | | |
| 7 | Washing w/ | 1.86 | 14 μg | 16 min |
| | alcohol | | | |
| 8 | Washing w/ | 1.92 | 16 μg | 16 min |
| | alcohol | | | |
| 9 | Wasbing w/ | 1.89 | 13 μg | 16 min |
| | alcohol | | | |

### Example 4

Purification of a range of DNA fragments from an aqueous solution; comparison of various binding buffers in regard to binding efficiency in relation to pH value of the binding buffer.

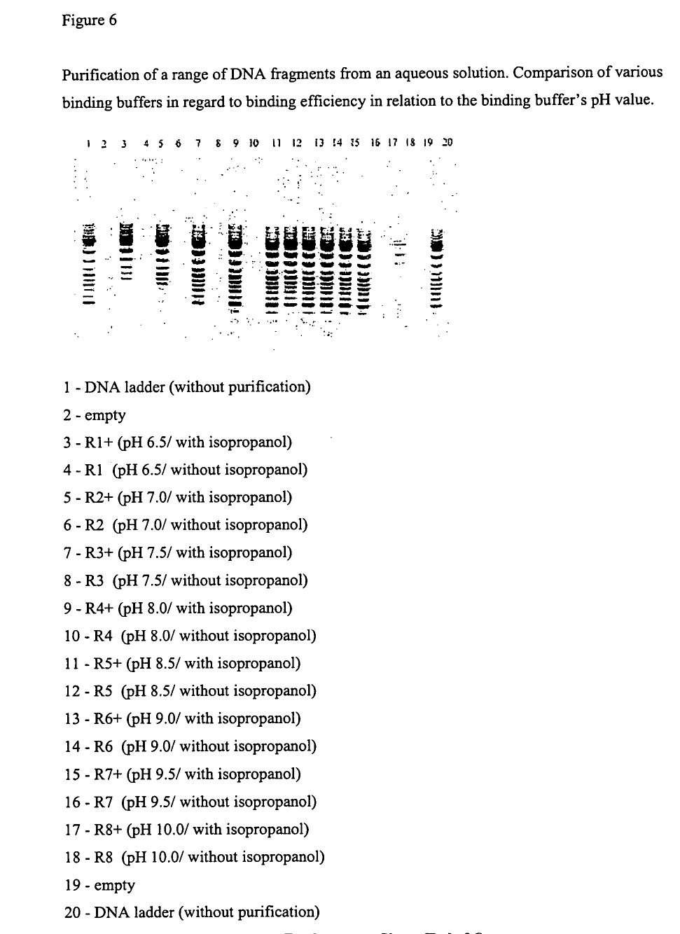

50 μl of an aqueous solution, containing 2 μg of a DNA ladder (Fermentas) were mixed with 130 μl of binding buffer (R1-R8), then transferred to a centrifuge column with a glass fiber fleece, centrifuged for 1 minute and finally the DNA was eluted from the column using 10 mM of tris-HCl. The isolated DNA fragments were then electrophoretically cut (FIG. 6).

Binding buffer: each containing 50 mM MgCl2 and optionally 20% isopropanol as well as 100 mM tris HCl with varying pH values.

R1+ (pH 6.5/with isopropanol)

R1 (pH 6.5/without isopropanol)

R2+ (pH 7.0/with isopropanol)

R2 (pH 7.0/without isopropanol)

R3+ (pH 7.5/with isopropanol)

R3 (pH 7.5/without isopropanol)

R4+ (pH 8.0/with isopropanol)

R4 (pH 8.0/without isopropanol)

R5+ (pH 8.5/with isopropanol)

R5 (pH 8.5/without isopropanol)

R6+ (pH 9.0/with isopropanol)

R6 (pH 9.0/without isopropanol)

R7+ (pH 9.5/with isopropanol)

R7 (pH 9.5/without isopropanol)

R8+ (pH 10.0/with isopropanol)

R8 (pH 10.0/without isopropanol)

1-34. (canceled)

35. A method for isolating nucleic acids from a solution by binding to a solid phase comprising the steps of:
a. providing a solution containing at least one nucleic acid;
b. combining said solution with additives containing multivalent cations and monovalent cations;
c. optionally combining said solution with an alcohol;
d. providing a solid carrier;
e. contacting said solution with said carrier and binding said at least one nucleic acid to said carrier;
f. removing said nucleic acid from said carrier using a washing buffer, wherein said washing buffer has a pH between 5 and 10 and:
i. does not comprise alcohol; or
ii. comprises an alcohol and at least one monovalent and/or multivalent cation.

36. The method of claim 35, wherein said multivalent and/or monovalent cations in said washing buffer are metallic cations.

37. The method of claim 35, wherein the ratio of monovalent cation to said multivalent cation is between about 9:1 to about 1:9.

38. The method of claim 35, wherein the final concentration of the salt components in solution is greater than 5 mMol.

39. The method of claim 35, wherein said alcohol is selected from the group consisting of ethanol or isopropanol.

40. The method of claim 35, further comprising addition of tris-HCl, or polyvinylpyrrilodone to the solution containing at least one nucleic acid.

41. The method of claim 35, wherein all carrier materials used to isolate chaotropic reagents are solid phase materials.

42. The method of claim 41, wherein said carrier materials are selected from the group consisting of glass fiber fleeces, silica membranes, membranes with functional groups that are equivalent to those on glass fiber fleeces or silica membranes, (SiO₂ suspensions, aerosols, and magnetized silica particles.

43. The method of claim 35, wherein said monovalent and multivalent salt components are present in ionically weak solutions.

44. The method of claim 35, further comprising the use of at least one member of the group consisting of water or water and tris-HCl is used as an elution buffer.

45. The method of claim 35, wherein said multivalent cations are divalent cations.

46. The method of claim 35, wherein said multivalent cations are Mg²⁺, Ca²⁺, Zn²⁺ or Mn²⁺.

47. The method of claim 35, wherein said monovalent cations are selected from the group consisting of NH₄⁺, Na⁺, or K⁺.

48. The method of claim 35, wherein the final concentration of salt components is >5 mMol.

49. The method of claim 35, wherein said alcohol is selected from the group consisting of ethanol, isopropanol, polyethylene glycol, and mixtures of the same.

50. The method of claim 35, wherein the pH value of the binding buffer is adjusted with tris-HCl.

51. The method of claim 47, wherein the pH value of the binding buffer without alcohol additive is between 8.5-9.5.

52. The method of claim 35, wherein the pH value of the binding buffer with alcohol additive is 5-9.5.

53. A test kit to isolate DNA from any base materials comprising:
a. an aqueous solution comprising monovalent and/or multivalent cations;
b. optionally, an alcohol;
c. optionally, a buffer;
d. a solid carrier produced centrifuge tube, wherein said solid carrier may be 96 or 384-gauged corrugated filtration plates;
e. washing buffer; and
f. elution buffer.

54. The test kit of claim 53, wherein said solid phase is selected from the group consisting of glass fiber fleece, glass membrane, silicon carrier, silicon dioxide, cut silicic acid, pyrgenous acid, magnetic silica particles, a membrane with functional groups and aerosol.

55. A test kit to isolate DNA from base materials comprising:
a. an aqueous solution comprising monovalent and multivalent cations, wherein said multivalent cations are divalent cations;
b. a solid phase, wherein said solid phase is a centrifuge tube component of 96 or 384-gauged corrugated filtration plates;
c. washing buffer, wherein said washing buffer does not comprise alcohol; and
d. elution buffer, wherein said elution buffer does not comprise alcohol.

56. The test kit of claim 55, wherein said solid phase is selected from the group consisting of glass fiber fleece, glass membrane, silicon carrier, aerosol, silicon dioxide, cut silicic acid, pyrgenous acid, magnetic silica particles and membranes with functional groups.
